(19) United States
(12) Patent Application Publication
(10) Pub. No.: US 2024/0165054 A1
(43) Pub. Date: May 23, 2024

(54) TREATMENT

(71) Applicant: Verona Pharma PLC, Cardiff (GB)

(72) Inventors: Tara Renae RHEAULT, Cary, NC (US); Kathleen RICKARD, Durham, NC (US); Thomas BENGTSSON, Lund (SE)

(21) Appl. No.: 18/424,452

(22) Filed: Jan. 26, 2024

Related U.S. Application Data
(63) Continuation of application No. PCT/GB2023/052084, filed on Aug. 7, 2023.
(60) Provisional application No. 63/502,977, filed on May 18, 2023.
(60) Provisional application No. 63/370,699, filed on Aug. 8, 2022.
(60) Provisional application No. 63/370,696, filed on Aug. 8, 2022.
(60) Provisional application No. 63/370,694, filed on Aug. 8, 2022.

(51) Int. Cl. A61K 31/137; A61K 9/00; A61K 31/519; A61K 47/02; A61K 47/26; A61P 11/08

(52) U.S. Cl. CPC A61K 31/137; A61K 9/0078; A61K 31/519; A61K 47/02; A61K 47/26; A61P 11/08

(57) ABSTRACT

The present invention relates to a compound for use in a method for decreasing the frequency and/or severity of chronic obstructive pulmonary disease (COPD) exacerbations in a patient suffering from COPD, wherein the compound is ensifentrine or a pharmaceutically acceptable salt thereof. The present invention also relates to a compound for use in a method for treating chronic obstructive pulmonary disease (COPD) in a patient, wherein: the compound is ensifentrine or a pharmaceutically acceptable salt thereof; and the patient is susceptible to COPD exacerbations.

### CROSS REFERENCE

This application is a bypass continuation of International Application No. PCT/GB2023/052084, filed on Aug. 7, 2023, which claims priority to U.S. Provisional Application No. 63/502,977, filed on May 18, 2023, U.S. Provisional Application No. 63/370,699, filed on Aug. 8, 2022, U.S. Provisional Application No. 63/370,696, filed on Aug. 8, 2022, and U.S. Provisional Application No. 63/370,694, filed on Aug. 8, 2022, each of which is incorporated herein by reference in its entirety.

### FIELD OF THE INVENTION

The present invention relates to decreasing the frequency and/or severity of chronic obstructive pulmonary disease (COPD) exacerbations in a patient suffering from COPD. The present invention also relates to the treatment of COPD in patients susceptible to COPD exacerbations.

### BACKGROUND OF THE INVENTION

Ensifentrine (N-(2-{(2E)-9,10-dimethoxy-4-oxo-2-[(2,4,6-trimethylphenyl)imino]-6,7-dihydro-2H-pyrimido[6,1-α]isoquinolin-3(4H)-yl}ethyl)urea; also known as RPL554) is a dual PDE3/PDE4 inhibitor and is described in WO 00/58308 A1.

As a combined PDE3/PDE4 inhibitor, ensifentrine has both bronchodilatory and anti-inflammatory activity and is useful in the treatment of respiratory disorders including chronic obstructive pulmonary disease (COPD). The chemical structure of ensifentrine is shown below.

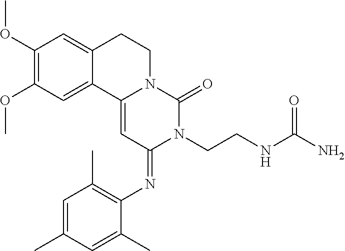 [SMILES: COc1cc2c(cc1OC)-c1c/c(=N\c3c(C)cc(C)cc3C)n(CCNC(N)=O)c(=O)n1CC2]

COPD is a progressive, long-term condition affecting a large number of people worldwide. Ongoing symptoms include breathlessness and cough. However, many COPD patients also suffer from occasional, temporary worsening of their symptoms (known as a COPD exacerbation). A COPD exacerbation typically involves an increase in breathlessness (dyspnea), the presence of excessive mucus (increased sputum volume), change in mucus colour (sputum purulence) and/or an increase in coughing.

Certain patient groups are particularly susceptible to COPD exacerbations. This includes patients suffering from additional respiratory conditions such as asthma and patients suffering from allergic conditions. Patients susceptible to COPD exacerbations have typically experienced COPD exacerbations previously.

COPD exacerbations can be severe and can limit the quality of life of COPD patients. Not all drugs which are disclosed for use in treating COPD are equally effective in reducing the severity and/or frequency of COPD exacerbations or time to first COPD exacerbation. It would therefore be advantageous for a COPD drug to be used which is particularly effective in reducing the severity and/or frequency of COPD exacerbations when there is a specific clinical need to address such exacerbations.

### SUMMARY OF THE INVENTION

It is a surprising finding of the present invention that ensifentrine is highly effective in reducing the frequency and/or severity of COPD exacerbations in COPD patients. In particular, ensifentrine has been found to lead to a more pronounced reduction in COPD exacerbations compared to many known treatments for COPD.

The invention accordingly provides a compound for use in a method for decreasing the frequency and/or severity of chronic obstructive pulmonary disease (COPD) exacerbations in a patient suffering from COPD, wherein the compound is ensifentrine or a pharmaceutically acceptable salt thereof. The invention also provides a compound for use in a method for increasing the time to first COPD exacerbation in a patient suffering from COPD, wherein the compound is ensifentrine or a pharmaceutically acceptable salt thereof.

Also provided by the invention is a compound for use in a method for treating chronic obstructive pulmonary disease (COPD) in a patient, wherein: the compound is ensifentrine or a pharmaceutically acceptable salt thereof; and the patient is susceptible to COPD exacerbations.

The invention also provides a method for decreasing the frequency and/or severity of chronic obstructive pulmonary disease (COPD) exacerbations in a patient suffering from COPD, the method comprising administering a therapeutically effective amount of a compound to the patient, which compound is ensifentrine or a pharmaceutically acceptable salt thereof.

The invention further provides a method of treating COPD in a patient, which method comprises administering a therapeutically effective amount of a compound which is ensifentrine or a pharmaceutically acceptable salt thereof to the patient, wherein the patient is susceptible to COPD exacerbations.

Further provided by the invention is use of a compound in the manufacture of a medicament for use in a method for decreasing the frequency and/or severity of chronic obstructive pulmonary disease (COPD) exacerbations in a patient suffering from COPD, wherein the compound is ensifentrine or a pharmaceutically acceptable salt thereof.

Also provided by the invention is use of a compound which is ensifentrine or a pharmaceutically acceptable salt thereof in the manufacture of a medicament for treating COPD, wherein the patient is susceptible to COPD exacerbations.

### BRIEF DESCRIPTION OF THE FIGURES

FIG. 1 shows the FEV₁ profile over 12 hours at Week 12.

### DETAILED DESCRIPTION OF THE INVENTION

The compound decreases the frequency and/or severity of chronic obstructive pulmonary disease (COPD) exacerbations in a patient suffering from COPD. The compound may decrease the frequency of COPD exacerbations. For instance, the patient may suffer two or fewer (for instance one or zero) COPD exacerbations per year while being treated with the compound, for instance as a maintenance therapy. The number of COPD exacerbations experienced by the patient per year during treatment with the compound may be one to three fewer than the number of COPD exacerbations experienced by the patient per year prior to treatment with the compound.

The compound may increase the time to a first COPD exacerbation in the patient. Accordingly, the patient may not have yet experienced a COPD exacerbation and the compound may increase the time until the patient experiences a first COPD exacerbation (i.e. the first COPD exacerbation is delayed). The compound may accordingly reduce the risk of COPD exacerbations in a COPD patient.

The COPD exacerbations typically comprise one or more of dyspnea (breathlessness), increased coughing, increased sputum volume, sputum purulence, wheezing, sore throat, a cold, and fever. Sputum purulence is a change in the colour of spontaneously expectorated samples from uncoloured to yellow-green. The COPD exacerbation may last for at least one day or at least two days.

A COPD exacerbation may comprise (A) worsening of two or more of the following major symptoms for at least two consecutive days: dyspnea, sputum volume and sputum purulence or (B) worsening of any one major symptom together with any one of the following minor symptoms for at least two consecutive days: sore throat, colds (nasal discharge and/or nasal congestion), fever (oral temperature >37.5° C.) without other cause and increased cough. For instance, a COPD exacerbation may comprise worsening of two or more of the major symptoms (dyspnea, sputum volume and sputum purulence) for at least two consecutive days.

A COPD exacerbation may be a moderate COPD exacerbation or a severe COPD exacerbation. A moderate exacerbation is defined as worsening symptoms of COPD (as defined above) requiring a minimum of three days of treatment with oral/systemic corticosteroids and/or antibiotics. A severe exacerbation is defined as worsening symptoms of COPD (as defined above) requiring in-patient hospitalization. The compound may reduce the severity of COPD exacerbations in a patient, and accordingly the compound may be for use in preventing severe COPD exacerbations in a patient. For instance, the patient may experience no severe COPD exacerbations in the year following first administration of the compound.

The compound may lengthen the time to a COPD exacerbation. For instance, it may increase the time to an exacerbation by two or more months.

The patient is typically susceptible to COPD exacerbations. Typically, a“patient susceptible to COPD exacerbations” is a patient suffering from one or more co-morbidities (other than COPD). Said patient is typically suffering from one or more disease or condition selected from asthma, pulmonary hypertension, bronchiectasis, allergy, lung cancer, chest infection, cystic fibrosis, pulmonary fibrosis, pneumonia, hay fever, allergic rhinitis, bronchitis, emphysema, adult respiratory distress syndrome (ARDS), interstitial lung disease or tuberculosis, optionally wherein the asthma is allergic asthma, steroid resistant asthma, severe asthma or paediatric asthma. The patient susceptible to COPD exacerbation may have a chronic bronchitis etiology, may have impaired lung function (such as 30 to 70% predicted FEV₁) or may have COPD symptoms despite use of long-acting muscarinic receptor antagonist (LAMA) or a long-acting beta-adrenergic receptor agonist (LABA) therapy.

For instance, the compound may be for use in treating COPD in a patient suffering from both COPD and asthma. The compound may be for use in treating COPD a patient suffering from both COPD and pulmonary hypertension. The compound may be for use in treating COPD in a patient suffering from both COPD and bronchiectasis.

Other risk factors for COPD exacerbation include: high serum immunoglobulin (Ig), previous tuberculosis, severe airflow obstruction, chest infection and one or more hospital admission for COPD exacerbations in the previous year.

In some cases, the patient has suffered one or more COPD exacerbations in the year preceding first administration of the compound. For instance, the patient may have suffered two or more COPD exacerbations in the year preceding first administration of the compound. The patient may for instance have suffered from at least one severe COPD exacerbation (i.e. requiring hospital treatment) in the preceding year. The patient may have suffered one or more COPD exacerbations in the six months preceding first administration of the compound, or one or more COPD exacerbations in the one month preceding first administration of the compound.

The patient may be male. The patient may be female. The patient may have an age of greater than or equal to 65 years. The patient may have an age of less than 65 years. The patient may be taking a background medication selected from one or more of a long-acting muscarinic antagonist (LAMA), a long-acting beta-agonist (LABA) and an inhaled corticosteroid (ICS).

The compound is ensifentrine or a pharmaceutically acceptable salt thereof. Pharmaceutically acceptable salts are well known to the skilled person. Typically, the compound is ensifentrine (i.e. ensifentrine free base).

The method typically comprises administering the compound to the patient by inhalation. A pharmaceutical composition comprising the compound and one or more pharmaceutically acceptable excipients or diluents is typically administered to the patient by inhalation, for instance by nebuliser, pressurised metered dose inhaler (pMDI) or dry powder inhaler (DPI).

Preferably, the method comprises administering the compound to the patient by inhalation from a nebuliser. Nebulisers aerosolise a liquid pharmaceutical composition into an aerosol that is inhaled into a patient's respiratory tract. Examples of nebulisers include a soft mist nebuliser, a vibrating mesh nebuliser, a jet nebuliser and an ultrasonic wave nebuliser. Suitable nebuliser devices include the Philips I-neb™ (Philips), the Philips SideStream (Philips), the AeroNeb ® (Philips), the Philips InnoSpire Go (Philips), the Pari LC Sprint (Pari GmbH), the AERxR™ Pulmonary Delivery System (Aradigm Corp) and the Pari LC Plus Reusable Nebuliser (Pari GmbH). The nebulizer may for instance be a PARI LC Sprint jet nebulizer with a PARI Vios® PRO Aerosol Delivery System PARI BOY® compressor. The compound may be inhaled via the nebuliser for from 1 to 15 minutes.

Typically, the method comprises administering the compound to the patient once, twice or three times per day, for instance twice or three times per day. The compound may be administered to the patient by inhalation once, twice or three times a day. Preferably the method comprises administering the compound to the patient by inhalation twice a day. The method may comprise administering a first dose of the compound in the morning (for instance within 3 hours following waking) and a second dose of the compound in the evening (for instance within 3 hours before bed). Typically, the morning and evening doses are administered from 10 to 14 hours apart, for instance about 12 hours apart.

The compound may be used in any suitable therapeutically effective amount. Typically, the daily dose of the compound is from 0.1 to 20 mg. Typically, the method comprises administering a total daily dose of the compound of from 0.5 to 10 mg. Preferably, the total daily dose of the compound (e.g. ensifentrine free base) is from 5 to 7 mg, for instance about 6 mg per day. As used herein, the term“about” may represent a variation of ±10% of the stated value. The total daily dose of the compound may be 6.0 mg.

Typically, the compound is administered twice a day in two separate doses which are the same or similar. For instance, the method may comprise administering the compound to the patient twice a day in a first dose of from 1 to 5 mg and a second dose of from 1 to 5 mg. Typically, the method may comprise administering the compound to the patient twice a day in a first dose of from 2 to 4 mg and a second dose of from 2 to 4 mg.

Preferably, the method comprises administering two doses of about 3 mg ensifentrine free base to the patient per day by inhalation. The method preferably comprises administering a dose of about 3 mg of the compound to the patient twice a day (3 mg BID) by inhalation. More preferably, the method comprises administering by nebuliser a dose of about 3 mg the compound to the patient twice a day. Each dose may be 3.0 mg free base ensifentrine administered by nebulizer.

The compound is typically used as a maintenance therapy. Typically, the method comprises administering the compound to the patient at least once per day for at least 8 weeks. The compound may be administered to the patient at least once per day for at least 16 weeks, preferably for at least 24 weeks. The compound may be administered daily to the patient for at least 1 year. The method may comprise administering the compound to the patient at least once every 24 hours, preferably at least twice every 24 hours, for at least 8 weeks, preferably for at least 16 weeks, more preferably for at least 24 weeks.

The compound is preferably administered as a suspension formulation, i.e. a suspension of particles comprising the compound in a diluent. The compound may alternatively be delivered as a dry powder, for instance a dry powder comprising particles comprising the compound and particles of a carrier such as lactose.

The method typically comprises administering an inhalable pharmaceutical composition comprising a suspension of particles of the compound in a diluent. The particles comprising the compound typically have a particle size distribution with a Dv50 of from 0.5 μm to 5.0 μm. The particles preferably have a Dv50 of from 1.0 μm to 2.0 μm.

Particle sizes are described herein by reference to the Dv50 value, which is the median particle size for a volume distribution. Thus, half the volume of the particles have diameters of less than the Dv50 value and half the volume of the particles have diameters of greater than the Dv50 value. This is a well-known manner in which to describe particle size distributions.

The technique used to measure the Dv50 values as stated herein is typically laser diffraction. The particle size distribution of the particles comprising the compound may be as measured by laser diffraction using a wet powder dispersion system. For instance, the particle size distribution can be measured by laser diffraction using a Malvem Spraytec in conjunction with a wet dispersion cell. Typically, the instrument parameters for the Malvem Spraytec are as follows:
particle—standard opaque particle;
refractive index Particle—1.50;
refractive index (imaginary)—0.50;
density of particle—1.00;
refractive index of dispersant—1.33;
controller unit—1000 RPM;
measurement type—timed;
initial sampling time—30s;
obscuration—20%-30%;
dispersant—1% Polysorbate 20 in deionised water.

The particles comprising the compound typically comprise ensifentrine (i.e. ensifentrine free base). The particles may comprise at least 90 wt % ensifentrine free base relative to the total weight of the particles. The particles may comprise at least 99 wt % ensifentrine. The particles may consist of ensifentrine.

The concentration of particles comprising the compound in the inhalable pharmaceutical composition is typically from 0.1 to 5.0 mg/mL, preferably from 0.1 to 2.5 mg/mL, more preferably from 1.0 to 2.0 mg/mL.

The inhalable pharmaceutical composition typically further comprises one or more tonicity adjusters, one or more buffers and one or more surfactants. The tonicity adjuster is typically sodium chloride.

Examples of buffers include a citrate buffer, a phosphate buffer, an acetate buffer, and a bicarbonate buffer. Preferably, the buffer is a phosphate buffer, for instance sodium dihydrogen phosphate dihydrate and/or disodium phosphate dihydrate.

Examples of surfactants include lecithin, oleic acid, polyoxyethylene glycol alkyl ethers (for instance PEG 300, PEG 600, PEG 1000, Brij 30, Brij 35, Brij 56, Brij 76 and Brij 97), polypropylene glycol (for instance PPG 2000), glucoside alkyl ethers, polyoxyethylene glycol octylphenol ethers, polyoxyethylene glycol alkylphenol ethers, glycerol alkyl esters, polyoxyethylene glycol sorbitan alkyl esters (polysorbates, for instance polysorbate 20, polysorbate 40, polysorbate 60 and polysorbate 80), sorbitan alkyl esters (for instance sorbitan monolaurate (Span 20), sorbitan monooleate (Span 80) and sorbitan trioleate (Span 85)), cocamide MEA, cocamide DEA, dodecyldimethylamine oxide, block copolymers of polyethylene glycol and polypropylene glycol (poloxamers), block copolymers of polyethylene glycol and polypropylene oxide (for instance Pluronic surfactants), polyvinyl pyrrolidone K25, polyvinyl alcohol, oligolactic acid, sodium dioctyl sulfosuccinate and polyethoxylated tallow amine (POEA).

Preferably, the one or more surfactants comprise a polysorbate and/or a sorbitan alkyl ester. The one or more surfactants may for instance comprise polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate) or polysorbate 80 (polyoxyethylene (20) sorbitan monooleate). The one or more surfactants may for instance comprise sorbitan monolaurate (Span 20), sorbitan monooleate (Span 80) or sorbitan trioleate (Span 85). Preferably, the sterile liquid vehicle comprises polysorbate 20 and/or sorbitan monolaurate (Span 20).

For instance, the method may comprise administering to the patient an inhalable liquid pharmaceutical composition comprising:
water;
particles consisting of ensifentrine free base at a concentration of from 0.1 to 20 mg/mL;
one or more tonicity adjusters at a total concentration of from 1.0 to 15 mg/mL;
one or more buffers at a total concentration of from 0.1 to 4 mg/mL; and
one or more surfactants at a total concentration of from 0.05 to 3 mg/mL.

The inhalable liquid pharmaceutical composition may comprise:
water;
particles consisting of ensifentrine free base at a concentration of from 0.5 to 6 mg/mL;
sodium chloride at a concentration of from 5 to 12 mg/mL;
sodium dihydrogen phosphate dihydrate at a concentration of from 0.3 to 2 mg/mL;
disodium phosphate dihydrate at a concentration of from 0.3 to 2 mg/mL;
polysorbate 20 at a concentration of from 0.1 to 1.5 mg/mL; and
sorbitan monolaurate at a concentration of from 0.01 to 0.5 mg/mL.

The compound may be used in combination with a second active agent. The compound may be administered separately or simultaneously with the second active agent. The patient may already be taking a second active agent as a background therapy for COPD. Alternatively, treatment with the second active agent may start at around the same time as treatment with the compound. The compound and the second active agent may be administered in a fixed combination.

The second active agent is typically a muscarinic receptor antagonist, a beta-adrenergic receptor agonist or an inhaled corticosteroid. The compound may accordingly be used in combination with muscarinic receptor antagonist, a beta-adrenergic receptor agonist or an inhaled corticosteroid. The second active agent may be a long-acting muscarinic receptor antagonist (LAMA) or a long-acting beta-adrenergic receptor agonist (LABA).

Examples of LAMAs include aclidinium, darotropium, tiotropium, glycopyrrolate and umeclidinium. Examples of LABAs include salmeterol, formoterol, indacaterol, vilanterol, olodaterol, abediterol and carmoterol. Examples of inhaled corticosteroids include beclomethosone, budesonide, fluticasone propionate, ciclesonide, mometasone and fluticasone furoate.

The patient may be using a beta-agonist (for instance salbutamol) as a rescue medication.

The invention is described in more detail by the following Example.

### EXAMPLES

### Study Design

A clinical study was conducted to determine the efficacy of ensifentrine in treating COPD compared with placebo. Ensifentrine was administered by nebuliser at a dose of 3 mg twice daily (BID) for 24 weeks. The study was a multi-centre, randomized, double-blind, parallel-group, placebo-controlled trial with around 800 patients and 5:3 randomization.

The study population included patients aged 40-80 years with moderate to severe COPD (FEV₁ 30%-70% p.n., FEV₁/forced vital capacity [FVC] ratio <0.7, with mMRC≥2). The randomization stratified (a) the use of stable background maintenance LAMA or LABA therapy use (approx. 50%. yes or no) and (b) cigarette smoking (current or former). Inhaled corticosteroid (ICS) maintenance therapy was permitted in up to 20% of patients under certain provisions.

The primary endpoint of the study was change from baseline in average FEV₁ area under the curve (AUC)0-12 h post-dose at week 12. Secondary endpoints of the study included: peak FEV₁ over 4 hours post-dose at Week 12; morning trough FEV₁ at Week 12; and other endpoints including moderate/severe COPD exacerbations frequency and time to first moderate or severe COPD exacerbation over 24 Weeks.

### Methods

COPD severity is derived as follows: mild: 80%<=FEV₁, moderate: 50%<=FEV₁<80% predicted, severe: 30%<=FEV₁<50% predicted and very severe: FEV₁<30% predicted, post bronchodilator dose at Screening.

Baseline FEV₁ is the mean of the two measurements taken before study medication on the day of first dosing, i.e. ≤40 minutes and just prior to dosing, pre-dose on day 1.

Average FEV₁ AUCO-12 h is defined as area under the curve over 12 hours of the FEV₁, divided by 12 hours.

Exacerbations were defined as worsening of 2 or more major symptoms (dyspnea, sputum volume, and sputum purulence) or worsening of any 1 major symptom together with any 1 of the following minor symptoms: sore throat, colds, fever without other cause, and increased cough or wheeze for at least 2 consecutive days. COPD exacerbations were considered to be of moderate severity if treatment with systemic corticosteroids and/or antibiotics was required and were considered to be severe if hospitalization was required.

### Formulation

The investigational product and placebo were provided in 2.5 mL unit dose format in an ampule and administered via a nebuliser. The formulation of the investigational product (ensifentrine suspension formulation) and placebo are shown in Table 1 below.

TABLE 1

| Constituent | Concentration (mg/mL) |
|---|---|
| Ensifentrine particles (RPL554) | 1.2 (for the active) or 0 (for Placebo) |
| Polysorbate 20 (Tween 20) | 0.50 |
| Sorbitan Monolaurate (Span 20) | 0.05 |
| Sodium Dihydrogen Phosphate Dihydrate | 0.744 |
| Disodium Hydrogen Phosphate Dihydrate | 0.853 |
| Sodium Chloride | 8.60 |
| Water | q.s. to 1 mL |

### Results

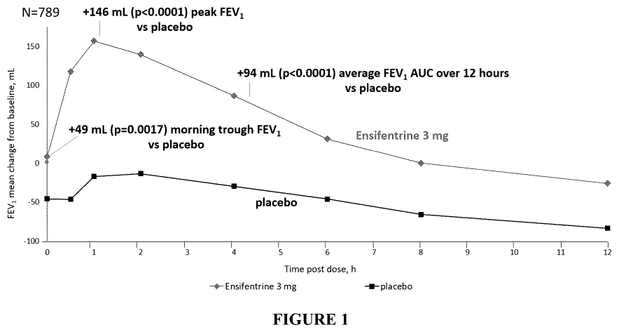

The primary endpoint of average FEV₁ (AUC)0-12 h at Week 12 was met. All subgroups showed improvement in lung function with ensifentrine that was statistically significant. The results are shown in Table 2 and FIG. 1.

TABLE 2

| Average FEV₁ (AUC)0-12 | Ensifentrine | |
|---|---|---|
| h post-dose at Week 12: | 3 mg | Placebo |
| Change from Baseline (CFB) | (n = 498) | (n = 291) |
| Observed N | 424 | 231 |
| Mean Baseline FEV₁, L | 1.3323 | 1.2316 |
| CFB Least Squares Mean, | 48.2 (30.0, 66.5) | −45.7 (−69.7, −21.6) |
| mL (95% CI) | | |
| Placebo-corrected CFB LS | 93.9 (64.5, 123.3) | — |
| Mean Diff., mL (95% CI) | | |
| p-value | <0.0001 | — |

Ensifentrine treatment resulted in approximately 40% reduction in relative risk of moderate or severe COPD exacerbation vs placebo (including 52% of patients on background bronchodilator medications).

### Comparison with Other Drugs

As a comparison, the reduction in the rate of COPD exacerbations achieved with other treatments (long-acting muscarinic antagonists (LAMAs), long-acting beta-agonist (LABAs), inhaled corticosteroids (ICSs) and PDE4 inhibitors) taken from the literature is shown in Table 4 below.

TABLE 4

| | | | | Rate Reduction |
|---|---|---|---|---|
| Drug | Class | Population | Rate Ratio | (=1-Rate Ratio) |
| Spiriva | LAMA | FEV₁ ≤ 60% predicted | 0.78 vs placebo | 22% |
| Tudorza | LAMA | 60% had at least one prior | 0.83 vs placebo | 17% |
| | | moderate or severe COPD | | |
| | | exacerbation within the past year | | |
| Breo 100/25 | LABA/ICS | at least one prior moderate or | 0.79 vs vilanterol | 21% |
| | | severe COPD exacerbation within | | |
| | | the past year | 0.66 vs vilanterol | 34% |
| Symbicort | LABA/ICS | at least one prior moderate or | 0.74 vs formoterol | 26% |
| | | severe COPD exacerbation within | | |
| | | the past year | 0.65 vs formoterol | 35% |
| Daliresp | PDE4 | At least one exacerbation in past | 0.85 vs placebo | 15% |
| | | year; LABA (44%) and LAMA | | |
| | | (35%) use allowed | 0.82 vs placebo | 18% |
| Tanimilast | PDE4 | at least one prior moderate or | Not reported | 13-28% all doses |
| | | severe COPD exacerbation within | | vs placebo |
| | | the past year | | |

Ensifentrine was found to cause a greater reduction in the frequency of COPD exacerbations than other treatments.

### Conclusion

It has been found that ensifentrine provides a statistically significant improvement in lung function in all subgroups of COPD patients in the study. In addition, ensifentrine has been found to be particularly effective in increasing the time to first exacerbation (i.e., reducing the risk of exacerbation) and reducing the frequency of COPD exacerbations.

What is claimed is:

1. A method for reducing frequency of an exacerbation of chronic obstructive pulmonary disease (COPD) in a human subject having COPD, wherein the exacerbation of COPD is a temporary worsening of one or more symptoms of COPD suffered by the human subject lasting at least two consecutive days, the method comprising administering to the human subject via inhalation two or more doses per day of a composition comprising a therapeutically effective amount of ensifentrine or a pharmaceutically acceptable salt thereof, wherein the frequency of the exacerbation of COPD is reduced by at least about 40% compared to the frequency of the exacerbation of COPD in an untreated human subject having COPD and not administered the composition.

2. The method of claim 1, wherein the composition is administered to the human subject as a maintenance therapy.

3. The method of claim 1, wherein the method reduces the frequency of the exacerbation of COPD by at least about 50% compared to the frequency of the exacerbation of COPD in the human subject prior to the administering to the human subject.

4. The method of claim 1, wherein the method further comprises administering to the human subject a muscarinic receptor antagonist, a beta-adrenergic receptor agonist, an inhaled corticosteroid, or a combination thereof.

5. The method of claim 4, wherein the beta-adrenergic receptor agonist is salbutamol.

6. The method of claim 1, wherein the one or more symptoms of COPD is selected from the group consisting of COPD related dyspnea, increased coughing, increased sputum volume, sputum purulence, wheezing, sore throat, cold, and fever.

7. The method of claim 6, wherein the exacerbation of COPD is dyspnea.

8. The method of claim 6, wherein the exacerbation of COPD is sputum purulence.

9. The method of claim 1, wherein the COPD is moderate COPD.

10. The method of claim 1, wherein the COPD is severe COPD.

11. The method of claim 1, wherein the human subject has one or more conditions selected from the group consisting of asthma, pulmonary hypertension, bronchiectasis, allergy, lung cancer, chest infection, cystic fibrosis, pulmonary fibrosis, pneumonia, hay fever, allergic rhinitis, bronchitis, emphysema, adult respiratory distress syndrome (ARDS), interstitial lung disease, tuberculosis, asthma, high serum immunoglobulin (Ig), previous tuberculosis, severe airflow obstruction, chest infection, and allergic conditions in addition to COPD.

12. The method of claim 1, wherein time to the exacerbation of COPD is increased by at least one month following the administering to the human subject compared to the time to the exacerbation of COPD prior to the administering to the human subject.

13. The method of claim 12, wherein the time to the exacerbation of COPD is increased by at least two months following the administering to the human subject.

14. The method of claim 1, wherein the administering to the human subject is twice per day in a first dose and a second dose.

15. The method of claim 14, wherein the first dose and the second dose are administered to the human subject in equal amounts.

16. The method of claim 14, wherein the first dose and the second dose are administered 10 to 14 hours apart.

17. The method of claim 14, wherein the therapeutically effective amount of the ensifentrine or the pharmaceutically acceptable salt thereof is 2 mg to 4 mg.

18. The method of claim 14, wherein the therapeutically effective amount of the ensifentrine or the pharmaceutically acceptable salt thereof is 3 mg.

19. The method of claim 1, wherein the composition is a suspension.

20. The method of claim 19, wherein the suspension comprises particles, wherein the particles comprise at least 90 weight percent ensifentrine relative to total weight of the particles.

21. The method of claim 19, wherein the suspension comprises particles, wherein the particles comprise at least 99 weight percent ensifentrine relative to total weight of the particles.

22. The method of claim 19, wherein the suspension comprises 3mg ensifentrine free base.

23. The method of claim 19, wherein the suspension comprises:
a. ensifentrine particles at a concentration of from 1 to 1.4 mg/mL;
b. polysorbate 20 (Tween 20) at a concentration of from 0.3 to 0.7 mg/mL;
c. sorbitan monolaurate (Span 20) at a concentration of from 0 to 0.1 mg/mL;
d. sodium dihydrogen phosphate dihydrate at a concentration of from 0.5 to 1 mg/mL;
e. disodium hydrogen phosphate dihydrate at a concentration of from 0.5 to 1 mg/mL;
f. sodium chloride at a concentration of 5 to 10 mg/mL; and
g. water.

24. The method of claim 19, wherein the suspension comprises:
a. 1.2 mg/ml ensifentrine;
b. 0.5mg/ml polysorbate 20;
c. 0.05 mg/ml sorbitan monolaurate;
d. 0.744 mg/ml sodium dihydrogen phosphate dihydrate;
e. 0.853 mg/ml disodium hydrogen phosphate dihydrate;
f. 8.6 mg/ml sodium chloride; and
g. water.

25. The method of claim 1, wherein the administering to the human subject is via a nebulizer.

26. The method of claim 1, wherein the administering to the human subject is via a pressurized metered dose inhaler (pMDI).

27. The method of claim 1, wherein the administering to the human subject is via a dry powder inhaler (DPI).

28. The method of claim 1, wherein the exacerbation of COPD is a moderate exacerbation as determined by the human subject requiring treatment with oral and/or systemic corticosteroids and/or antibiotics as a result of the exacerbation of COPD.

29. The method of claim 1, wherein the exacerbation of COPD is a severe exacerbation as determined by the human subject requiring hospitalization as a result of the exacerbation of COPD.
